(19) United States
(12) Patent Application Publication
(10) Pub. No.: US 2007/0244440 A1
(43) Pub. Date: Oct. 18, 2007

(54) Medical device with expandable tip

(75) Inventors: Dharmendra Pal, Wilmington, MA (US); Thomas A. Osborne, Bloomington, IN (US)

Correspondence Address: BRINKS HOFER GILSON & LIONE/CHICAGO/COOK, PO BOX 10395, CHICAGO, IL, 60610, US

(73) Assignee: Cook Incorporated, Bloomington, IN (US)

(21) Appl. No.: 11/728,528

(22) Filed: Mar. 26, 2007

Related U.S. Application Data
(60) Provisional application No. 60/786,440, filed on Mar. 28, 2006.

(51) Int. Cl. A61M 29/00; A61M 5/178; A61M 25/00

(52) U.S. Cl. 604/164.13; 604/523; 604/104

(57) ABSTRACT

A catheter for use with at least two different sizes of wire guides is described. The catheter includes a wire guide lumen sized to receive a first wire guide of a first diameter. The catheter may also include a tip lumen that extends in a distal direction from a first opening in communication with the wire guide lumen to a second opening. The first opening is sized to receive the first wire guide, and the second opening is sized to receive a second wire guide of a smaller diameter than the first wire guide. The catheter also includes one or more longitudinal expansion features capable of radially expanding the tip lumen to receive a wire guide of a diameter up to the first diameter through the second opening. An adapter that may be attached to the catheter by a medical professional is also described.

### RELATED APPLICATIONS

The present patent document claims the benefit of the filing date under 35 U.S.C. § 119(e) of Provisional U. S. Patent Application Ser. No. 60/786,440, filed Mar. 28, 2006, which is hereby incorporated by reference.

### TECHNICAL FIELD

The present invention relates generally to medical devices and more particularly to catheters.

### BACKGROUND

Medical delivery catheters are well known in the art of minimally invasive surgery for the introduction and delivery of fluids and devices to sites inside a patient's body. For example, a catheter may be used to introduce radiopaque contrast media or to position and deploy an expandable stent within a body vessel.

Typically, a catheter is threaded over a wire guide and directed to an intraluminal site of interest. Different sizes of wire guides may be employed in different medical procedures and/or for different body vessels or treatment locations. A catheter is in turn designed to accommodate, or receive, a wire guide of a particular diameter.

Some procedures may benefit from the use of wire guides having different diameters. It may be desirable, for example, for a physician to access a body vessel using a relatively small diameter wire guide, and later in the procedure exchange the small diameter wire guide for a larger diameter wire guide that provides higher stiffness and better pushability. As an example, a physician may want to guide a balloon catheter to a stenosis in a coronary artery using a 0.018 inch wire guide after accessing the artery with a 0.014 inch wire guide. To do so, the physician may direct the balloon catheter, which is sized to receive a 0.018 inch wire guide, over the 0.014 inch wire guide, and then remove the 0.014 inch wire guide and exchange it for a 0.018 inch wire guide.

In such situations, the difference between the sizing of the catheter lumen (0.018 inch) and the initial wire guide diameter (0.014 inch) may result in the exposure of a ledge at the inside edge of the catheter tip as the catheter is maneuvered along the initial wire guide through the vessel. Such a ledge may rub against the vessel wall as the catheter is directed to the treatment site. Thus, it is preferable that gaps between the outer surface of the wire guide and the inner surface of the catheter lumen be minimized to reduce rubbing against the vessel wall as the catheter is advanced.

It would therefore be desirable to design a catheter that could be properly and safely used with more than one size of wire guide.

### SUMMARY

Described herein are a catheter and intraluminal delivery system that may accommodate more than one size of wire guide. Also described are methods of medical treatment using the catheter and intraluminal delivery system. A medical device including an expandable tip is also described.

The catheter described in this disclosure includes a wire guide lumen sized to receive a first wire guide of a first diameter. The catheter also includes a tip lumen that extends from a first opening to a second opening in a distal direction. The first opening is in communication with the wire guide lumen and is sized to receive the first wire guide. The second opening is sized to receive a second wire guide of a smaller diameter than the first wire guide. The catheter may also include one or more longitudinal expansion features capable of radially expanding the tip lumen to receive a wire guide of a diameter up to the first diameter through the second opening.

The intraluminal delivery system described herein includes a catheter having a wire guide lumen sized to accommodate a first wire guide. The system also includes an adapter engageable with the catheter. The adapter has an adapter lumen sized to accommodate a second wire guide that is smaller in diameter than the first wire guide.

According to one embodiment, the method of medical treatment includes introducing an initial wire guide into a body lumen, and passing a catheter over the initial wire guide. The catheter may include a tube portion having a wire guide lumen sized to accommodate a first wire guide of a first diameter, and a tip portion distal to the tube portion. The tip portion has a tip lumen extending in a distal direction from a first opening in communication with the wire guide lumen to a second opening. The first opening is sized to accommodate the first wire guide and the second opening is sized to accommodate a second wire guide of a second diameter smaller than the first diameter. The tip portion includes one or more longitudinal expansion features capable of radially expanding the tip lumen to accommodate a wire guide of a diameter up to the first diameter through the second opening. The initial wire guide has a diameter no larger than the first diameter and no smaller than the second diameter. The initial wire guide is removed from the catheter and the body lumen and a next wire guide is inserted into the body lumen and through the catheter. The next wire guide has a diameter no larger than the first diameter and no smaller than the second diameter, and a diameter larger than the diameter of the initial wire guide.

According to another embodiment, a method of medical treatment includes providing a catheter having a wire guide lumen sized to accommodate a first wire guide, and providing an adapter engageable with the catheter. The adapter has an adapter lumen sized to accommodate a second wire guide smaller in diameter than the first wire guide. The adapter is engaged with the catheter. The second wire guide is inserted into a body lumen and directed to a treatment site, and then the catheter is passed over the second wire guide for transfer to the treatment site.

The medical device with the expandable tip includes a tube portion having a first lumen of a first diameter, and a tip portion distal to the tube portion. The tip portion has an opening at a distal end thereof, and the opening has a second diameter smaller than the first diameter. The device includes one or more longitudinal pleats in a wall of the tip portion, where each longitudinal pleat comprises two folds in the wall. The longitudinal pleats are capable of radially and circumferentially expanding the opening to increase the second diameter. The longitudinal pleats are not present in the tube portion.

According to one embodiment, the two folds of the longitudinal pleat are aligned in a substantially circumferential direction when the longitudinal pleated is in a folded configuration. The longitudinal pleats may have a tapered circumferential width that increases from a proximal end of the pleat to a distal end of the pleat at the opening.

According to an alternative embodiment, the two folds of the longitudinal pleat are aligned in a substantially radial direction when the longitudinal pleat is in a folded configuration. The longitudinal pleats may have a tapered radial height that increases from a proximal end of the pleat to a distal end of the pleat at the opening.

According to another embodiment, the longitudinal pleats may have a length of about 20 mm or less.

According to another embodiment, the longitudinal pleats may have a length that is less than about 5% of an entire length of the medical device.

### BRIEF DESCRIPTION OF THE DRAWINGS

FIG. 1 is a sectional view of a distal portion of a catheter according to one embodiment;

FIGS. 2A-2D are sectional views of a distal portion of a catheter receiving a first wire guide, where the first opening of the catheter has been sized to accommodate the first wire guide;

FIGS. 3A-3B are sectional views of a distal portion of a catheter receiving a second wire guide, where the second opening of the catheter has been sized to accommodate the second wire guide;

FIGS. 4A-4B are sectional views of a distal portion of a catheter receiving a third wire guide which is smaller in diameter than the first wire guide, but larger than the second;

FIGS. 5A-5B are side and front views of a distal portion of a catheter which show the presence of longitudinal slits, according to another embodiment;

FIGS. 6A-6B are side and front views of a distal portion of a catheter which show the presence of longitudinal depressions, according to another embodiment;

FIGS. 7A-7B are perspective views of a distal portion of a catheter including one longitudinal pleat in folded and partially unfolded configurations, according to an embodiment in which folds of the longitudinal pleat are aligned in a circumferential direction in the folded configuration;

FIGS. 8A-8B are perspective views of a distal portion of a catheter including a coupled pleat structure in folded and partially unfolded configurations, according to an embodiment in which folds of the longitudinal pleats are aligned in a circumferential direction in the folded configuration;

FIG. 8C is a front view of a distal portion of a catheter including two coupled pleat structures in a folded configuration according to the embodiment of FIGS. 8A-8B;

FIG. 8D is a perspective view of a distal portion of a catheter including a coupled pleat structure in a folded configuration, according to another embodiment in which folds of the longitudinal pleats are aligned in a circumferential direction in the folded configuration;

FIGS. 9A-9B are perspective views of a distal portion of a catheter including a number of adjacent longitudinal pleats in folded and partially unfolded configurations, according to an embodiment in which folds of the longitudinal pleats are aligned in a radial direction in the folded configuration;

FIG. 9C is a front view of a distal portion of a catheter including two sets of adjacent longitudinal pleats according to the embodiment of FIGS. 9A-9B;

FIG. 9D is a perspective view of a distal portion of a catheter including a number of adjacent longitudinal pleats in a folded configuration, according to another embodiment in which folds of the longitudinal pleats are aligned in a radial direction in the folded configuration;

FIGS. 10A-10B are front views according to two embodiments of a distal portion of a catheter showing alternating arrangements of longitudinal slits and depressions about the circumference;

FIG. 11 is a front view of a distal portion of a catheter showing alternating arrangements of longitudinal slits and depressions in a longitudinal direction at four locations about the circumference, according to another embodiment; and

FIG. 12 is a side sectional view of a distal portion of an intraluminal delivery system, according to one embodiment.

FIG. 13 is a side sectional view of a distal portion of an intraluminal delivery system, according to another embodiment.

### DETAILED DESCRIPTION

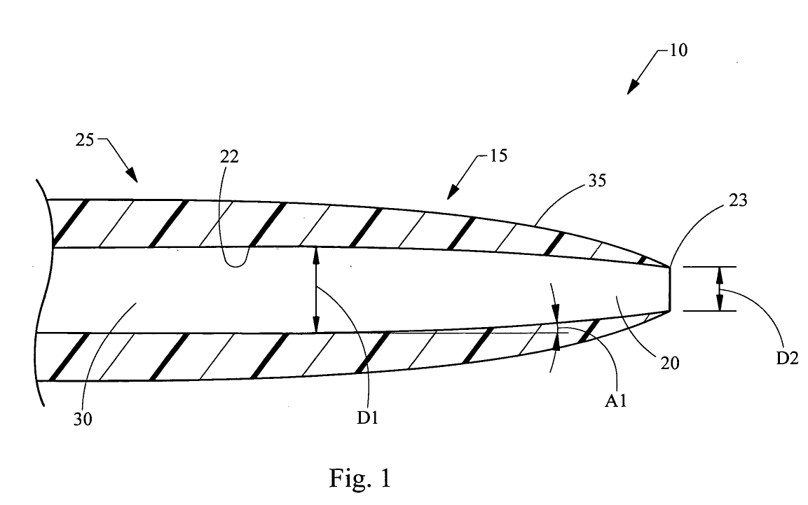

FIG. 1 shows a sectional view of a distal portion of a catheter 10 according to one embodiment. The catheter 10 includes a tip portion 15 having a tip lumen 20 and a tube portion 25 having a wire guide lumen 30 with a substantially uniform tube diameter.

The tip lumen 20 extends in a distal direction from a first opening 22 having a larger lumen diameter D1 to a second opening 23 having a smaller lumen diameter D2. Therefore, the tip lumen has a taper A1. The first opening 22 is in direct communication with the wire guide lumen 30, and the larger lumen diameter D1 is substantially the same as the tube diameter. The angle of the taper A1 may range from about one degree to about 30 degrees. Preferably, the angle of the taper A1 may range from about 5 degrees to about 15 degrees.

The catheter 10 may be sized to receive a first wire guide 40 through the first opening 22. For example, the catheter 10 may be sized to accommodate a first wire guide 40 having a nominal diameter of 0.035 inch through the first opening 22 of diameter D1. The catheter 10 may also be sized to receive a second wire guide 40′ of a smaller diameter through the second opening 23. For example, the catheter 10 may be sized to accommodate a second wire guide 40′ of a nominal 0.014 inch in diameter through the second opening 23 of diameter D2.

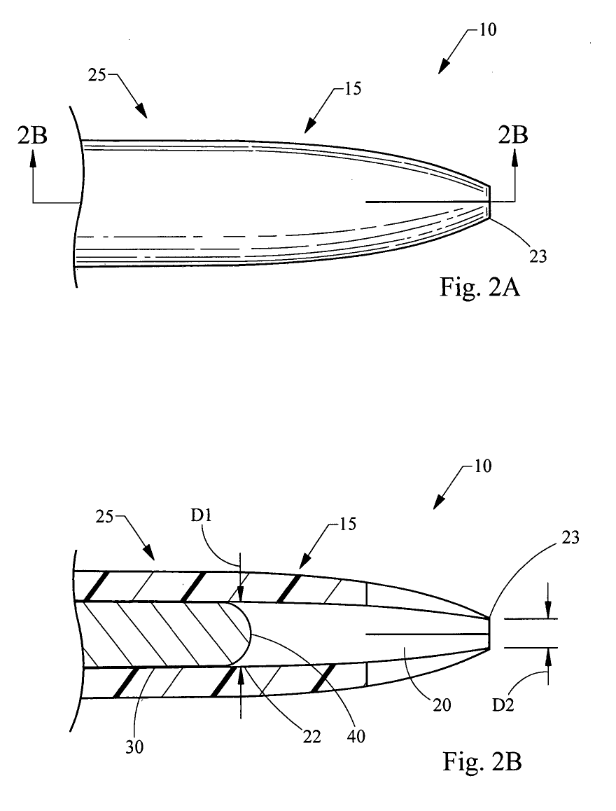

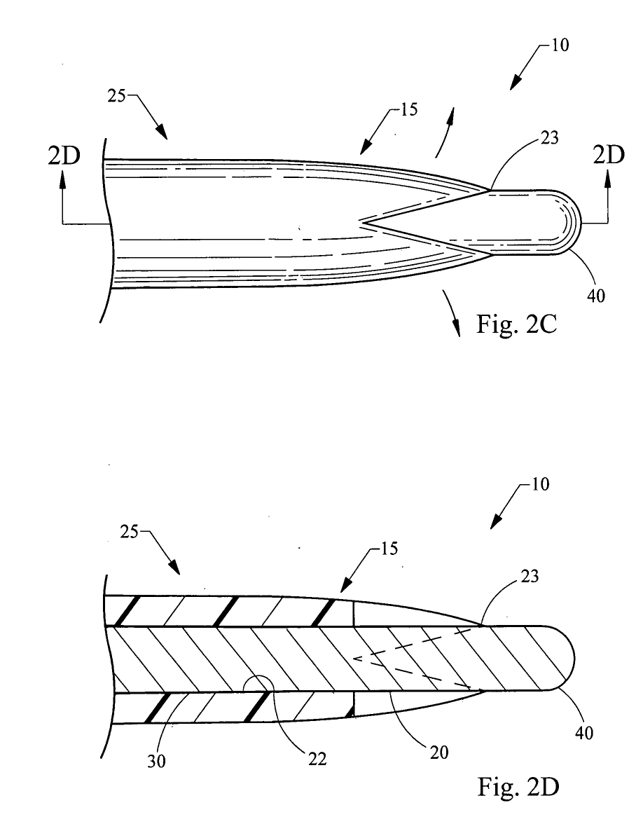

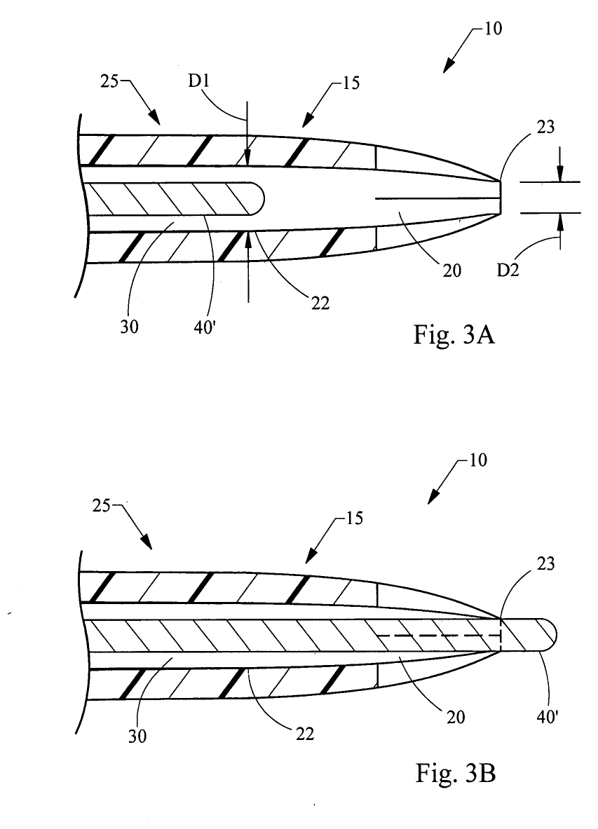

FIGS. 2A-2B, 3A-3B and 4A-4B are sectional views of a distal portion of a catheter 10 according to one embodiment. In these figures, the catheter 10 is shown accommodating or receiving wire guides of three different sizes. In FIGS. 2A-2B, the catheter 10 receives a first wire guide 40 of a first diameter. The first opening 22 of the catheter 10 has been sized to accommodate the first wire guide 40. In FIGS. 3A-3B, the catheter 10 receives a second wire guide 40′ of a second diameter. The second opening 23 of the catheter 10 has been sized to accommodate the second wire guide 40′. In FIGS. 4A-4B, the catheter 10 receives a third wire guide 40″ of a third diameter, where the third diameter is larger than the second diameter, but smaller than the first.

Referring first to FIG. 2A, the first wire guide 40 may be accommodated within the wire guide lumen 30 and the first opening 22. However, the diameter of the first wire guide 40 is larger than the diameter of the second opening 23. Longitudinal expansion features (not visible in this figure) present in the tip portion 15 of the catheter 10, however, allow the tip lumen 20 to expand radially to accommodate the first wire guide 40 through the second opening 23, as shown in FIG. 2B. As a result, the second opening 23 expands when the first wire guide passes through. The longitudinal expansion features may be longitudinal slits, longitudinal depressions, longitudinal pleats, or a combination thereof, and are described in detail below.

FIGS. 4A-4B show the catheter 10 accommodating a third wire guide 40″ which is smaller in diameter than the first wire guide 40, but larger than the second wire guide 40′. Similar to the example of FIG. 2B, the third wire guide 40″ may be accommodated due to the presence of longitudinal expansion features in the tip portion 15 of the catheter 10. In this case, the longitudinal expansion features allow the tip lumen 20 to expand partially in a radial direction as the wire guide passes through the second opening 23.

In FIGS. 3A-3B, the second wire guide 40′ is sized to correspond to the second opening 23 of the tip portion 15 of the catheter 10. Thus, the second wire guide 40′ may be directed through the second opening 23 without expanding the tip lumen 20.

Preferably, the first opening 22 and the second opening 23 may each be sized to accommodate a wire guide of a standard size. (It is to be understood that references to the sizing of the second opening 23 are predicated on the second opening being in an initial or unexpanded state for the sizing.) For example, the first opening 22 may be sized to accommodate a wire guide having a nominal diameter of 0.035 inch. The second opening 23 may be sized to accommodate a wire guide having a nominal diameter of 0.018 inch. In this case, one or more wire guides having a nominal diameter within the range of from 0.018 inch to 0.035 inch may be advantageously used with the catheter. In another example, the first opening 22 may be sized to accommodate a wire guide having a nominal diameter of 0.018 inch, and the second opening 23 may be sized to accommodate a wire guide having a nominal diameter of 0.014 inch. In this case, one or more wire guides having a nominal diameter within the range of from 0.014 inch to 0.018 inch may be advantageously used with the catheter.

Preferably, the first opening 22 may be sized to accommodate a wire guide having a nominal diameter in the range of from 0.010 inch to 0.038 inch, and the second opening 23 may be sized to accommodate a wire guide having a nominal diameter in the range of from 0.008 inch to 0.035 inch. More preferably, the first opening 22 may be sized to accommodate a wire guide having a nominal diameter in the range of from 0.021 inch to 0.038 inch, and the second opening 23 may be sized to accommodate a wire guide having a nominal diameter in the range of from 0.010 inch to 0.018 inch.

To accommodate a wire guide of a particular size, each of the first and second openings 22, 23 may generally be sized to provide about 0.001 inch of slack around the perimeter of the wire guide. For example, to accommodate a first wire guide 40 of 0.035 inch in nominal diameter, the first opening 22 may have a larger lumen diameter D1 of about 0.037 inch. In another example, to accommodate a second wire guide 40′ of 0.014 inch in nominal diameter, the second opening 23 may have a smaller lumen diameter D2 about 0.016 inch.

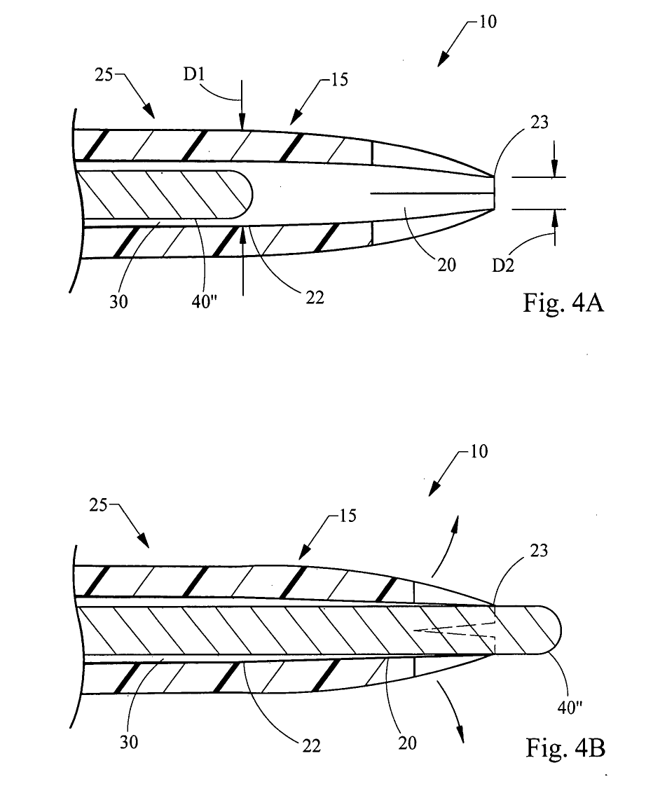

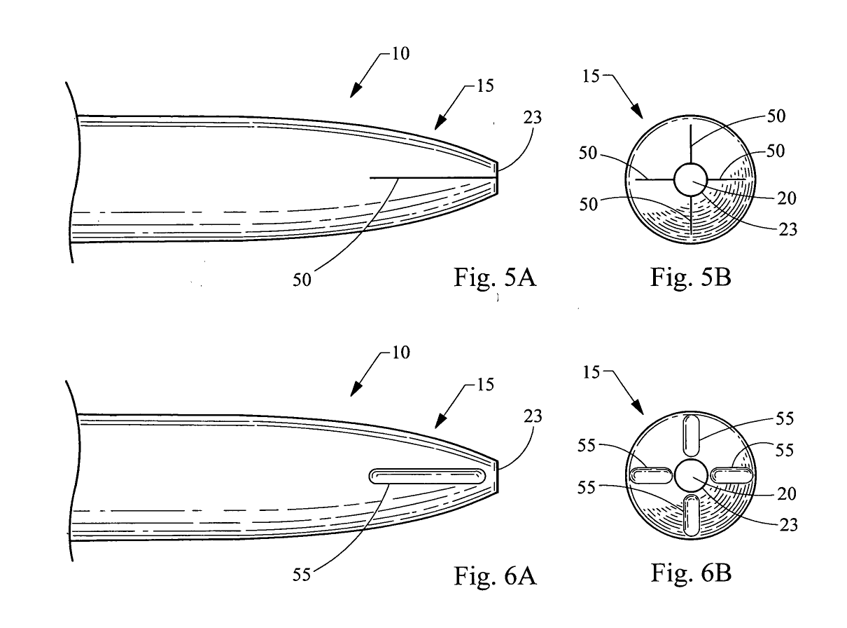

FIGS. 5A-5B and FIGS. 6A-6B show side and front views of a distal portion 15 of a catheter 10 according to two embodiments.

According to the embodiment shown in FIGS. 5A-5B, the distal portion of the catheter 10 may include one or more longitudinal slits 50 that pass through the wall thickness of the tip portion 15. For example, the distal portion may include two, three, four, five, six, seven or eight longitudinal slits 50 disposed about the circumference of the tip lumen 20. The slits 50 may be arranged symmetrically about the circumference of the tip lumen 20. Alternatively, the slits 50 may be arranged asymmetrically about the circumference of the tip lumen 20 if desired.

The presence of one or more longitudinal slits 50 may render the tip portion 15 expandable in a radial direction. For example, if a wire guide having a diameter larger than the smaller lumen diameter D2 is passed through the catheter 10, the slits 50 may facilitate radial expansion of the tip lumen 20 to accommodate the wire guide.

The longitudinal slits 50 may extend in a longitudinal direction over substantially the entire length of the tip portion 15. Each of the longitudinal slits 50 may have a length, for example, in the range of from about 1 mm to about 20 mm. Alternatively, the longitudinal slits 50 may extend over only a part of the length of the tip portion 15.

According to one embodiment in which the catheter 10 is a long-wire catheter, as will be described below, the length of each of the longitudinal slits 50 may be less than about 5% of the entire length of the catheter, where the entire length includes the length of the tube portion 25 and the length of the distal tip portion 15.

According to the embodiment shown in FIG. 6A-6B, the distal portion of the catheter 10 may include one or more longitudinal depressions 55 that pass partially through the wall thickness of the tip portion 15. For example, the tip portion 15 may include two, three, four, five, six, seven or eight longitudinal depressions 55 disposed about the circumference of the tip lumen 20. The depressions 55 may be arranged symmetrically about the circumference of the tip lumen 20. Alternatively, the depressions 55 may be arranged asymmetrically about the circumference of the tip lumen 20.

The depressions 55 may pass through at least about 25% of the wall thickness. Alternatively, the depressions 55 may pass through at least about 50% of the wall thickness. The depressions 55 may also pass through at least about 60%, 75% or 90% of the wall thickness.

The presence of one or more longitudinal depressions 55 may render the tip portion 15 more pliable or expandable in a radial direction. For example, if a wire guide having a diameter larger than the smaller lumen diameter D2 is passed through the catheter 10, the depressions 55 may facilitate radial expansion of the tip lumen 20 to accommodate the wire guide.

The longitudinal depressions 55 may extend in a longitudinal direction over substantially the entire length of the tip portion 15. Each of the longitudinal depressions 55 may have a length, for example, in the range of from about 1 mm to about 20 mm. Alternatively, the longitudinal depressions 55 may extend over only a portion of the length of the tip portion 15. According to one embodiment, the depressions 55 may have a width ranging from about 0.1 mm to about 1 mm.

According to one embodiment in which the catheter 10 is a long-wire catheter, as will be described below, the length of each of the longitudinal depressions 55 may be less than about 5% of the entire length of the catheter 10, where the entire length includes the length of the tube portion 25 and the length of the distal tip portion 15.

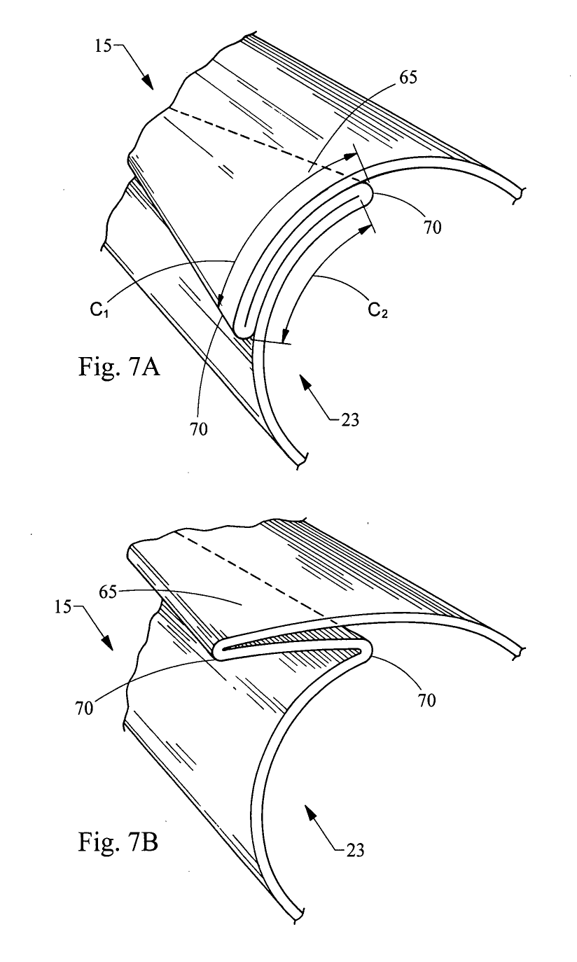

According to another embodiment shown in FIGS. 7A and 7B, the catheter 10 may include a longitudinal pleat 65 in the wall of the tip portion 15. The longitudinal pleat 65 preferably includes two folded portions 70. The pleat 65 is shown in a folded configuration in FIG. 7A and in a partially unfolded configuration in FIG. 7B. According to this embodiment, the folded portions 70 of the longitudinal pleat 65 are aligned in a circumferential direction in the folded configuration.

According to one embodiment, the tip portion 15 includes only one longitudinal pleat 65. Alternatively, the tip portion may include more than one longitudinal pleat 65, such as, for example, two, three, four, five, six or more longitudinal pleats 65. The longitudinal pleats 65 may be disposed symmetrically or asymmetrically about the circumference of the tip portion 15.

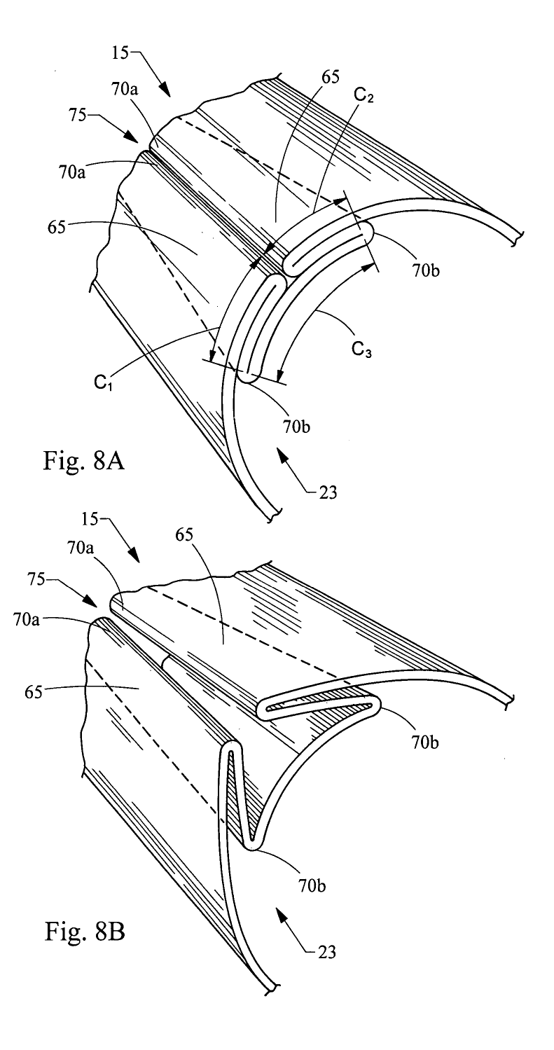

Referring to FIGS. 8A and 8B, the tip portion 15 may include a coupled pleat structure 75. The coupled pleat structure 75 includes two adjacent longitudinal pleats 65 and four folded portions 70. The four folded portions 70 are aligned in a circumferential direction when the coupled pleat structure is in a folded configuration, as shown in FIG. 8A. A first folded portion 70a of one of the two pleats 65 is adjacent and oppositely disposed to a first folded portion 70a of the other of the two pleats 65, and a second folded portion 70b of the one pleat 65 is oppositely disposed to a second folded portion 70b of the other pleat 65. FIG. 8B shows the coupled pleat structure 75 of FIG. 8A in a partially unfolded configuration.

According to one embodiment, the first folded portions 70a are disposed farther outward in a radial direction than are the second folded portions 70b, as shown in FIGS. 8A and 8B. This configuration may be referred to as an “outwardly coupled pleat structure.” However, it is also possible that the first folded portions 70a of the coupled pleat structure 75 may be disposed farther inward in a radial direction than are the second folded portions 70b. Such an “inwardly coupled pleat structure” is shown, for example in FIG. 8D.

According to one embodiment, the tip portion 15 includes only one coupled pleat structure 75. Alternatively, the tip portion 15 may include more than one coupled pleat structure, such as, for example, two, three, four or five coupled pleat structures 75. FIG. 8C shows a front view of a tip portion 15 including two coupled pleat structures 75 comprising of a total of four longitudinal pleats 65 and eight folded portions 70. The longitudinal pleats 65 of this embodiment are outwardly coupled together as shown in FIGS. 8A and 8B, with two adjacent folds 70 disposed in opposition to each other on an outer part of the tip portion 15 and two folds 70 disposed in opposition to each other on an inner part of the tip portion 15. A tip portion 15 including more than one coupled pleat structure 75 may include only outwardly coupled pleat structures or only inwardly coupled pleat structures; alternatively, the tip portion 15 may include a combination of outwardly and inwardly coupled pleat structures. The coupled pleat structures 75 may be disposed symmetrically or asymmetrically about the circumference of the tip portion 15.

If the tip portion 15 includes at least three longitudinal pleats 65, then a combination of pleat arrangements (single and coupled structures) is possible. In other words, one or more of the longitudinal pleats 65 may be singly arranged as shown in FIGS. 7A-7B and two or more of the longitudinal pleats 65 may be coupled together according to an embodiment of the coupled pleat structure 75 shown in FIGS. 8A-8D.

The presence of one or more longitudinal pleats 65 may render the tip portion 15 radially and circumferentially expandable. For example, if a wire guide having a diameter larger than the smaller lumen diameter D2 is passed through the catheter 10, the one or more pleats 65 may unfold to facilitate radial and circumferential expansion of the tip lumen 20 to accommodate the wire guide. Each longitudinal pleat 65 may have a tapered circumferential width that increases from a proximal end of the pleat 65 to a distal end of the pleat 65 at the second opening 23, as shown for example in FIG. 8C. For a single longitudinal pleat 65, the maximum amount of circumferential expansion corresponds approximately to the sum of the circumferential widths C₁ and C₂ between the folds 70 at the distal end of the tip portion 15, as shown, for example, in FIG. 7A. For a coupled pleat structure 75, the maximum amount of circumferential expansion corresponds approximately to the sum of the circumferential widths C₁, C₂, and C₃ between the folds 70 at the distal end of the tip portion 15, as shown in FIG. 8B. An approximate value of the diameter of the tip portion 15 when expanded may be calculated using the well known geometric relationship between diameter and circumference. Preferably, the diameter of the tip portion 15 when expanded is at least equivalent to D1.

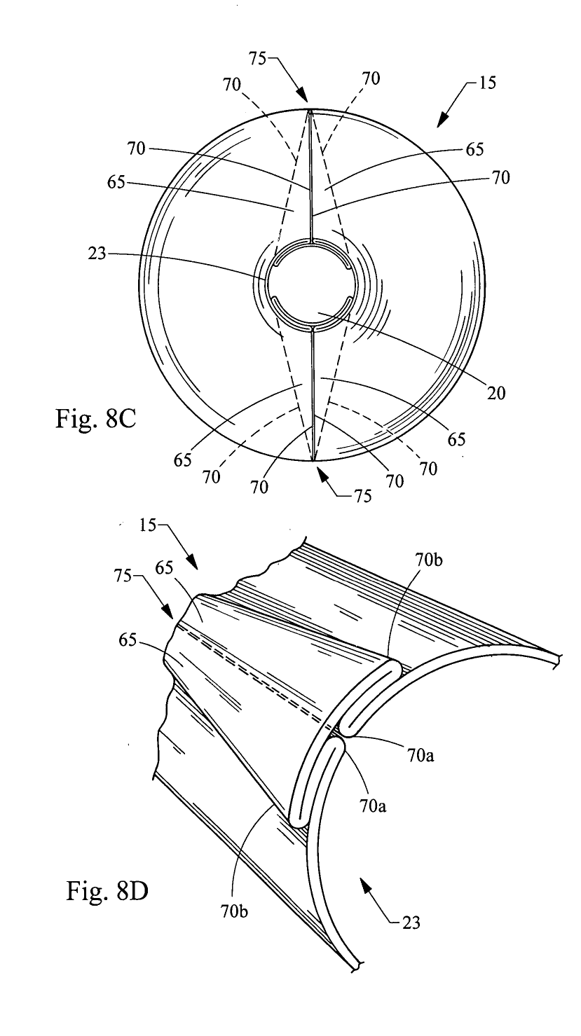

FIGS. 9A and 9B show an alternative embodiment of a pleated tip structure that may also render the tip portion 15 radially and circumferentially expandable. As shown in FIG. 9A, the tip portion 15 may include one or more longitudinal pleats 65′ having folded portions 70′ aligned in a radial direction when the pleats 65′ are in a folded configuration. Each longitudinal pleat 65′ preferably includes two folded portions 70′ and has a radial height H₁ at the distal end of the tip portion 15. According to this embodiment, the tip portion 15 preferably includes a plurality of adjacent longitudinal pleats 65′.

Referring to FIG. 9B, which shows the longitudinal pleats 65′ in a partially unfolded configuration, the amount of circumferential expansion achievable for a tip portion 15 including the longitudinal pleats 65′ depends on the number of pleats and the radial height H₁. The radial height of each longitudinal pleat 65′ is preferably tapered along the length of the tip portion 15, as shown in FIG. 9C. In other words, the radial height increases from a first value at or near a proximal end of the pleat 65′ to the value H₁ at a distal end of the pleat 65′ at the second opening 23.

The longitudinal pleats 65′ may be disposed such that outer ends 71 of the folds 70′ in the wall of the tip portion 15 do not extend outside the outer circumference of the wall in the folded configuration, as shown in FIG. 9A. According to this embodiment, the pleats 65′ do not increase the profile of the tip 15. Alternatively, the outer ends 71 of the folds 70′ may be disposed such that they extend outside the outer circumference of the wall in the folded configuration, as shown in FIG. 9D.

Preferably, the longitudinal pleats 65, 65′ extend over substantially the entire length of the tip portion 15. Each of the longitudinal pleats 65, 65′ may have a length in the range of, for example, from about 1 mm to about 20 mm.

According to one embodiment in which the catheter 10 is a long-wire catheter, as will be described below, the length of each of the longitudinal pleats 65, 65′ may be less than about 5% of the entire length of the catheter 10, where the entire length includes the length of the tube portion 25 and the length of the distal tip portion 15.

In contrast to the embodiment in which the tip portion 15 includes longitudinal slits 50 or depressions 55, the longitudinal pleats 65, 65′ do not penetrate or pass through the wall of the tip portion 15. However, at least a portion of the wall of the tip portion 15 of the catheter 10 may have a reduced thickness compared to that of the tube portion 25 to accommodate the folds 70, 70′ of the pleats 65, 65′ without substantially increasing the profile of the tip portion 15.

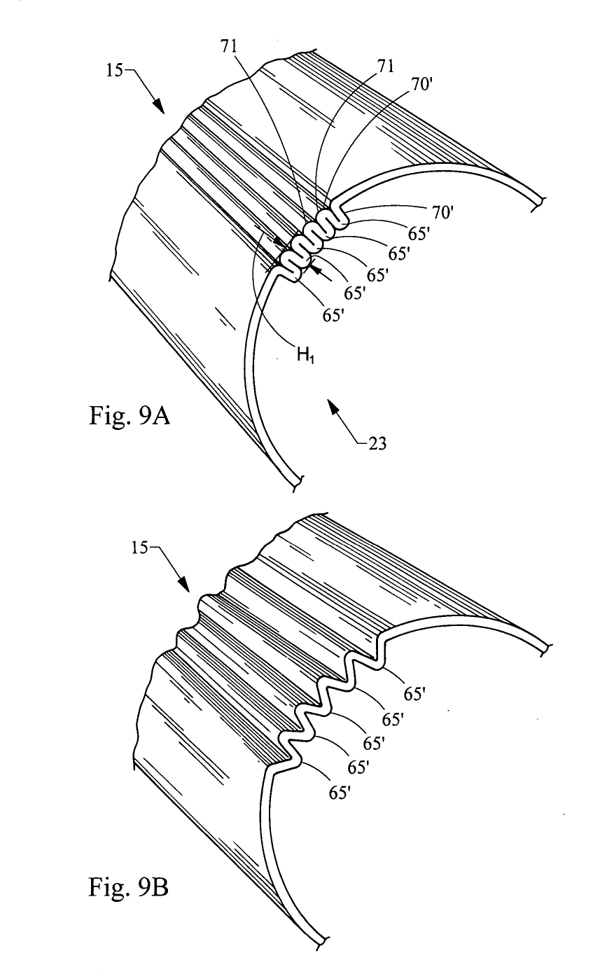

The tip portion 15 may include a combination of longitudinal slits 50, depressions 55, and pleats 65, 65′. According to one embodiment, the one or more longitudinal slits 50 and one or more depressions 55 may be arranged alternately about the circumference of the tip portion 15, as shown, for example, in FIG. 10A. In this figure, two slits 50 are arranged alternately with two depressions 55 about the circumference. In FIG. 10B, as another example, three slits 50 are disposed alternately with three depressions 55. Alternatively, any number of slits 50, depressions 55, and pleats may be combined. Symmetric arrangements about the circumference are preferred, but asymmetric arrangements are also possible.

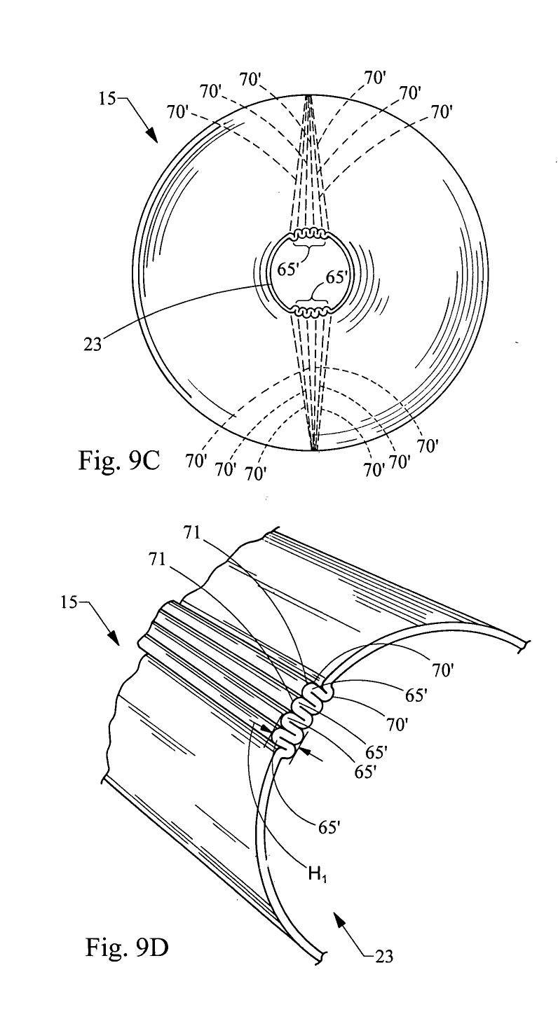

According to another embodiment, one or more slits 50 and one or more depressions 55 may be arranged alternately in a longitudinal direction at one or more circumferential positions. For example, as shown in FIG. 11, a slit 50 may extend in the longitudinal direction from the distal end of the catheter 10 to a distance partway along the tip portion 15 at, for example, each of the twelve o'clock, three o'clock, six o'clock and nine o'clock circumferential positions. Also, a depression 55 may extend in the longitudinal direction proximal to each of the slits 50, partway along the tip portion 15 and also at each of the twelve o'clock, three o'clock, six o'clock and nine o'clock circumferential positions. Alternatively, other circumferential positions and other alternating arrangements of slits 50 and depressions 55 in the longitudinal direction may be used.

The tip portion 15 may have a tapered outer surface 35 in addition to a tapered lumen. The outer surface 35 may extend from a larger outer diameter to a smaller outer diameter in the distal direction, and thus have a taper. The angle of the taper may range from about 1 degree to about 30 degrees. According to one embodiment, the wall thickness of the tip portion may gradually decrease in the distal direction.

A catheter 10 as described herein may be made from one or more polymers, such as, for example, a polyamide (e.g., nylon), a fluorocarbon (e.g., polytetrafluoroethylene (PTFE) or fluoroethylene-propylene (FEP)), polyether block amide (PEBA), polyolefin, polyimide, polyurethane, or polyvinyl chloride (PVC). The catheter 10 may include a metallic reinforcement structure, such as a coiled or braided wire, extending through part or all of the tube portion 25 of the catheter 10 to impart column strength. The catheter 10 may also include a coating, such as, for example, a hydrophilic coating, on a surface of the catheter 10.

The tip portion 15 of the catheter 10 may be integrally formed with the tube portion 25. For example, the tube and tip portions 25, 15 of the catheter 10 may be fabricated by extrusion as a single extruded tube, followed by a forming operation to impart a taper to one end of the tube. The forming operation may further provide one or more longitudinal expansion features on the tip portion 15, such as depressions 55. The forming operation may also provide a thinned section that may be folded to form longitudinal pleats 65 in the tip portion 15.

Extrusion of the tube may be carried out using extrusion methods and equipment known in the art. Forming of the tip portion 15 may be carried out by using any forming method known in the art, such as, for example, molding. A molding operation may entail, for example, inserting a pin into the lumen of the extruded tube to maintain the dimensions of the lumen during forming, and then placing the extruded tube into a bottom section of a mold having a tapered design. The mold may further include one or more protrusions corresponding in dimensions and position to the dimensions and position of any desired longitudinal depressions 55 in the tip portion 15 of the catheter 10. A mandrel or top section of the mold may then be lowered to apply pressure to the tube and heat may be applied. Upon application of heat and pressure, a tapered tip portion 15 corresponding to the tapered design of the mold may be formed.

Alternatively, the tip portion 15 of the catheter 10 may be extruded and formed separately from the tube portion 25 and then attached to the tube. The tip portion 15 may be attached to the catheter 10 by any known attachment method, including, for example, adhesive or thermal bonding, frictional force, or a locking mechanism.

The one or more longitudinal expansion features may be formed during molding, as described above, and/or by modification of the tip portion 15 after molding. For example, the longitudinal expansion features may be formed by cutting partially or all the way through the thickness of the tip portion 15 using a blade or a laser. In the case of longitudinal pleats 65, a thinned section in the wall of the tip portion 15 may be formed by molding, and then the thinned section may be folded to form the pleats 65, 65′.

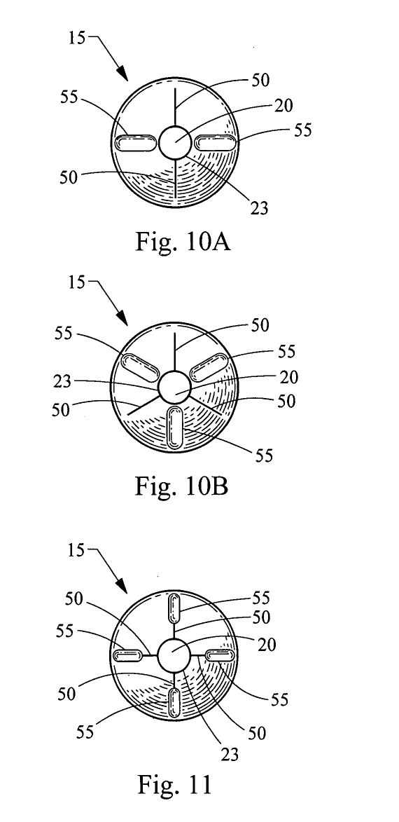

FIG. 12 shows a sectional view of an embodiment of an intraluminal delivery system for use with wire guides of different sizes. According to this embodiment, the delivery system 100 may include a catheter 110 having a distal portion 115 and a wire guide lumen 120 extending through at least the distal portion 115. The wire guide lumen 120 may have a substantially uniform diameter. The wire guide lumen 120 may be sized to accommodate a first wire guide 40.

The delivery system may include an adapter 125 engageable with the catheter 110 by a medical professional. The adapter 125 may have an adapter lumen 130 of a substantially uniform diameter. The diameter of the adapter lumen 130 may be smaller than the diameter of the catheter lumen 120. The adapter lumen 130 may be sized to accommodate a second wire guide 40′ which is smaller in diameter than the first wire guide 40.

Preferably, the wire guide lumen 120 and the adapter lumen 130 each may be sized to accommodate a wire guide of a standard size. For example, the wire guide lumen 120 may be sized to accommodate a wire guide having a nominal diameter of 0.035 inch. The adapter lumen 130 may be sized to accommodate a wire guide having a nominal diameter of 0.018 inch. In another example, the wire guide lumen 120 may be sized to accommodate a wire guide having a nominal diameter of 0.018 inch, and the adapter lumen 130 may be sized to accommodate a wire guide having a nominal diameter of 0.014 inch.

Preferably, the wire guide lumen 120 may be sized to accommodate a wire guide having a nominal diameter in the range of from 0.010 inch to 0.038 inch, and the adapter lumen 130 may be sized to accommodate a wire guide having a nominal diameter in the range of from 0.008 inch to 0.035 inch. More preferably, wire guide lumen 120 may be sized to accommodate a wire guide having a nominal diameter in the range of from 0.021 inch to 0.038 inch, and the adapter lumen 130 may be sized to accommodate a wire guide having a nominal diameter in the range of from 0.010 inch to 0.018 inch.

To accommodate a wire guide of a particular size, each of the wire guide lumen 120 and adapter lumen 130 may generally be designed to provide about 0.001 inch of slack around the perimeter of the wire guide. For example, to accommodate a first wire guide 40 having a nominal diameter of 0.018 inch, the wire guide lumen 120 may be about 0.020 inch in diameter. In another example, to accommodate a second wire guide 40′ having a nominal diameter of 0.014 inch, the adapter lumen 130 may be about 0.016 inch in diameter.

The adapter 125 may have an outer surface 135 which extends from a larger diameter to a smaller diameter in the distal direction, and thus has a taper. The angle of the taper may range from about 1 degree to about 30 degrees. Preferably, the angle of the taper may range from about 5 degrees to about 15 degrees. As a result of the taper, the wall thickness of the adapter 125 may gradually decrease in the distal direction.

The distal portion 115 of the catheter 110 may include a counterbore 145 within the wire guide lumen 120 to expand the opening of the lumen 120 to accommodate an adapter 125 having a diameter larger than that of the wire guide lumen 120, as shown in FIG. 10. Such a configuration may provide the advantage of a smoother transition between the diameter of the wire guide lumen 120 and the diameter of the adapter lumen 130. The counterbore may also provide a proximal stop or ledge 150 to aid in manual placement of the adapter 125 within the wire guide lumen 120.

The adapter 125 may have a length which is much smaller than the length of the catheter. For example, according to one embodiment in which the catheter 110 is a long-wire catheter, as will be described below, the length of the adapter 125 may be less than about 5% of the length of the catheter 110. Generally, the length of the adapter 125 may be in the range of from about 5 mm to about 20 mm.

Preferably, the adapter 125 may be made from a polymer, such as, for example, a polyamide (e.g., nylon), a fluorocarbon (e.g., polytetrafluoroethylene (PTFE) or fluoroethylene-propylene (FEP)), polyether block amide (PEBA), polyolefin, polyimide, polyurethane, or polyvinyl chloride (PVC). The adapter 125 may be fabricated by molding, such as described above, or by any other forming method known in the art.

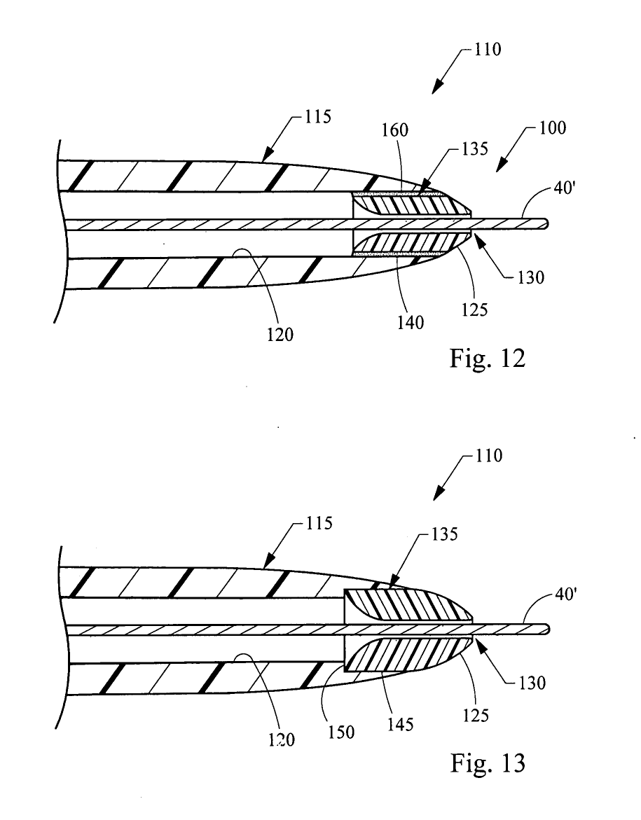

The engagement of the adapter 125 with the catheter 110 may be carried out by a medical professional. To engage the adapter 125 with the catheter 110, any known attachment method, including, for example, adhesive or thermal bonding, frictional force, or a locking mechanism, may be used. According to one embodiment, an adhesive 160 may be applied to the outer surface 135 of the adapter 125 and/or the wall of the wire guide lumen 120 prior to insertion of the adapter 125 into the wire guide lumen 120, as shown in FIG. 12. Alternatively, the adapter 125 may be pushed into the wire guide lumen 120 and retained in place by friction between the outer surface 135 of the adapter and the wall of the wire guide lumen 120, as shown in FIG. 13. Preferably, a portion of the outer surface 135 of the adapter 125 may be accommodated within the wire guide lumen 120 to facilitate engagement of the adapter 125 with the catheter 110.

The catheter 110 may have a long-wire or short-wire configuration. A long-wire catheter is one in which a wire guide lumen extends from a distal end of the catheter through substantially the entire length of the catheter. A short-wire catheter (sometimes referred to as a rapid exchange catheter) has a wire guide lumen extending from the distal end through only a portion of the length of the catheter.

The catheter 110 may further have more than one lumen, such as, for example, two lumens or three lumens. An additional lumen may be provided, for example, for inflation of a balloon or the delivery of contrast fluid. The additional lumen may or may not extend through the entire length of the catheter.

A catheter or intraluminal delivery system as described herein may be used with wire guides of different diameters. In some procedures, for example, it may be beneficial to access a body vessel using a relatively small diameter wire guide, and later in the procedure exchange the small diameter wire guide for a larger diameter wire guide that provides higher stiffness and better pushability. As an example, a physician may want to guide a balloon catheter to a stenosis in a coronary artery using a 0.018 inch wire guide after accessing the artery with a 0.014 inch wire guide. In another example, a physician may, for reasons of convenience, wish to use a catheter sized to receive a particular wire guide with a wire guide of a different size. A catheter or intraluminal delivery system according to the embodiments described herein may be sized to accommodate more than one size of wire guide and thus may be useful in these circumstances. Advantageously, the catheter or intraluminal delivery system may be directed through a vessel without causing damage to the vessel wall.

A method of medical treatment according to one embodiment may include first introducing an initial wire guide into a body lumen. Next, a catheter 10 sized to receive a first wire guide 40 of a large diameter and a second wire guide 40′ of a small diameter, as described herein according to various embodiments, may be passed over the initial wire guide. Preferably, the initial wire guide may have a diameter no larger than the large diameter and no smaller than the small diameter. Next, the initial wire guide may be removed from the body lumen, and a next wire guide may be inserted into the body lumen and passed through the catheter. Preferably, the next wire guide may have a diameter no larger than the large diameter and no smaller than the small diameter. Depending on the diameter of the initial wire guide and the diameter of the next wire guide, one or more longitudinal expansion features disposed within the tip portion 15 of the catheter 10 may allow the tip lumen 20 of the catheter 10 to expand radially as the initial and next wire guides pass through the second opening 23. According to one embodiment, the diameter of the next wire guide is larger than the diameter of the initial wire guide.

A method of medical treatment according to another embodiment may include providing a catheter 110 as described herein having a wire guide lumen 120 sized to accommodate a first wire guide 40. An adapter 125 engageable with the catheter 110 and having an adapter lumen 130 sized to accommodate a second wire guide 40′ may also be provided. The second wire guide 40′ may be smaller in diameter than the first wire guide 40. The adapter 125 may be engaged with the catheter 110 by a medical professional. The second wire guide 40′ may be inserted into a body lumen and directed to a treatment site. The catheter 110 including the adapter 125 may then be passed over the second wire guide 40′ for transfer to the treatment site.

Although the present invention has been described in considerable detail with reference to certain embodiments thereof, other embodiments are possible without departing from the present invention. The spirit and scope of the appended claims should not be limited, therefore, to the description of the preferred embodiments contained herein. All embodiments that come within the meaning of the claims, either literally or by equivalence, are intended to be embraced therein. Furthermore, the advantages described above are not necessarily the only advantages of the invention, and it is not necessarily expected that all of the described advantages will be achieved with every embodiment of the invention.

1. An intraluminal catheter for use with wire guides of different sizes, comprising:
a tube portion having a wire guide lumen sized to accommodate a first wire guide of a first diameter;
a tip portion distal to the tube portion, the tip portion having a tip lumen extending in a distal direction from a first opening in communication with the wire guide lumen to a second opening, the first opening being sized to accommodate the first wire guide and the second opening being sized to accommodate a second wire guide of a second diameter smaller than the first diameter,
wherein the tip portion comprises one or more longitudinal expansion features capable of radially expanding the tip lumen to accommodate a wire guide of a diameter up to the first diameter through the second opening.

2. The intraluminal catheter according to claim 1, wherein the longitudinal expansion features comprise longitudinal slits extending through a wall of the tip portion.

3. The intraluminal catheter according to claim 1, wherein the longitudinal expansion features comprise longitudinal depressions extending partially through a wall of the tip portion.

4. The intraluminal catheter according to claim 1, wherein the longitudinal expansion features comprise longitudinal pleats disposed in a wall of the tip portion, each longitudinal pleat comprising two folds in the wall.

5. The intraluminal catheter according to claim 4, wherein the longitudinal expansion features comprise a coupled pleat structure including two longitudinal pleats, a first fold of one of the pleats being adjacent and oppositely disposed to a first fold of the other pleat, wherein the first folds are aligned in a circumferential direction.

6. The intraluminal catheter according to claim 1, comprising two or more longitudinal expansion features disposed about a circumference of the tip portion.

7. The intraluminal catheter according to claim 6, wherein the two or more longitudinal expansion features are disposed symmetrically about a circumference of the tip portion.

8. The intraluminal catheter according to claim 1, wherein a wall of the tip portion gradually decreases in thickness from the first opening to the second opening.

9. The intraluminal catheter according to claim 1, wherein the tip portion is integrally formed with the tube portion.

10. The intraluminal catheter according to claim 1, wherein the tip portion is formed separately from the tube portion and attached to the tube portion.

11. The intraluminal catheter according to claim 1, comprising two or more longitudinal expansion features disposed symmetrically about a circumference of the tip portion, the longitudinal expansion features comprising at least one of one or more longitudinal slits extending through a wall of the tip portion and one or more longitudinal depressions extending partially through the wall of the tip portion, wherein the wall of the tip portion gradually decreases in thickness from the first opening to the second opening.

12. An intraluminal delivery system, comprising:
a catheter having a wire guide lumen sized to accommodate a first wire guide; and
an adapter engageable with the catheter, the adapter having an adapter lumen sized to accommodate a second wire guide smaller in diameter than the first wire guide.

13. The intraluminal delivery system according to claim 12, wherein the adapter lumen has a substantially uniform diameter.

14. The intraluminal delivery system according to claim 12, wherein a wall thickness of the adapter gradually decreases in a distal direction.

15. The intraluminal delivery system according to claim 12, wherein the adapter is engageable with the catheter by a medical professional.

16. A method of medical treatment, comprising:
introducing an initial wire guide into a body lumen;
passing a catheter over the initial wire guide, the catheter comprising:
a tube portion having a wire guide lumen sized to accommodate a first wire guide of a first diameter;
a tip portion distal to the tube portion, the tip portion having a tip lumen extending in a distal direction from a first opening in communication with the wire guide lumen to a second opening, the first opening being sized to accommodate the first wire guide and the second opening being sized to accommodate a second wire guide of a second diameter smaller than the first diameter,
wherein the tip portion comprises one or more longitudinal expansion features capable of radially expanding the tip lumen to accommodate a wire guide of a diameter up to the first diameter through the second opening;
removing the initial wire guide from the catheter and the body lumen, the initial wire guide having a diameter no larger than the first diameter and no smaller than the second diameter; and
inserting a next wire guide into the body lumen and through the catheter, the next wire guide having a diameter no larger than the first diameter and no smaller than the second diameter.

17. The method of medical treatment according to claim 16, wherein the initial wire guide is smaller in diameter than the next wire guide.

18. A medical device with an expandable tip, the medical device comprising:
a tube portion having a first lumen of a first diameter;
a tip portion distal to the tube portion, the tip portion having an opening at a distal end of the tip portion, the opening having a second diameter smaller than the first diameter; and
one or more longitudinal pleats in a wall of the tip portion, each longitudinal pleat comprising two folds in the wall, the longitudinal pleats being capable of radially and circumferentially expanding the opening to increase the second diameter,
wherein the longitudinal pleats are not present in the tube portion.

19. The medical device of claim 18, comprising a coupled pleat structure including two longitudinal pleats, a first fold of one of the pleats being adjacent and oppositely disposed to a first fold of the other pleat, wherein the first folds are aligned in a circumferential direction.

20. The medical device of claim 18, comprising a plurality of longitudinal pleats, wherein the folds of the pleats are aligned in a radial direction.
